(19) United States
(12) Patent Application Publication
(10) Pub. No.: US 2004/0058331 A1
(43) Pub. Date: Mar. 25, 2004

(54) Blood testing method, testing chip, and testing device

(75) Inventor: Kiwamu Akagi, Saitama (JP)

Correspondence Address: COVINGTON & BURLING, ATTN: PATENT DOCKETING, 1201 PENNSYLVANIA AVENUE, N.W., WASHINGTON, DC, 20004-2401, US

(73) Assignees: TUM Gene, Inc., Chiba-shi (JP); Kiwamu Akagi, Ageo-shi (JP)

(21) Appl. No.: 10/252,011

(22) Filed: Sep. 23, 2002

(51) Int. Cl. C12Q 1/68; G01N 31/00; G01N 33/574

(52) U.S. Cl. 435/6; 435/7.23

(57) ABSTRACT

The present invention provides a blood testing method capable of accurately and easily performing quantitative analysis of the progression of tumors and the therapeutic reactivity, without placing any heavy burden on a patient, by detecting a mutation factor derived from tumor cells and a normal factor with no mutation in a sample containing human plasma with the mutation factor, and by calculating a relative ratio between the mutation factor amount and the normal factor amount.

### BACKGROUND OF THE INVENTION

1. Field of the Invention

The present invention relates to a method for testing blood taken from a human body in order to grasp the progression of tumors and the therapeutic effect, and also relates to a testing chip and a testing device for practicing such method.

2. Description of the Related Art

Image diagnosis and tumor markers have been used as a testing method for measuring the progression of malignant tumors. In many cases, however, the progression of malignant tumors cannot be evaluated by the analysis of the image diagnosis or tumor markers.

However, in clinical practices, precise evaluation of the progression of cancer and the therapeutic effect is important in determining a therapeutic policy. An easy testing method that does not place any heavy burden on patients is desired to be established.

Against this background, testing methods for detecting minor substances in the blood have been suggested. As attention is given to the fact that cancer causes mutations to accumulate on genomes (DNA), these methods are used to detect DNA derived from cancer cells. Due to the fact that compared to a normal individual, the amount of DNA in a cancer patient's blood plasma or blood serum is increased, it has been suggested that a testing method to conduct measurement of the DNA concentration in blood plasma or blood serum be utilized (Stroun M. et al., “Isolation and Characterization of DNA from the Plasma of Cancer Patients,” Eur J Cancer Clin Oncol, 1987 June, 23(6) 707-712; Yumi Hayashi et al., “Identification of Plasma DNA and Application to the Detection of K-ras Gene Mutation,” Clinical Pathology, 2000, 547-553). Moreover, it has been reported that if DNA with mutation inherent in cancer is found in blood plasma, DNA derived from cancer cells exists in the blood plasma (deKok J B et al., “Detection of Tumor DNA in Serum of Colorectal Cancer Patients,” Scand J Clin Lab Invest, 1997 Nov., 57 (7) 601; Esteller M. et al., “Detection of Aberrant Promoter Hypermethylation of Tumor Suppressor Genes in Serum DNA from Non-small Cell Lung Cancer Patients,” Cancer Res, 1999 August, 159(1) 67-70).

The above-mentioned method of measuring DNA concentration in the blood plasma or blood serum of cancer patients is to detect an increase in the DNA concentration as cytolysis causes DNA to flow into the blood. However, the original DNA concentration varies among individuals and also changes due to factors other than cancer. Accordingly, there is a problem in that it is actually difficult to conduct the test quantitatively as a tumor marker.

Moreover, the above-described method for detecting DNA with mutation derived from cancer cells can determine whether cancer exists or not, but can be considered nothing more than a qualitative test of DNA with mutation.

### SUMMARY OF THE INVENTION

The present invention aims to solve the above-described problems. It is the object of this invention to provide a blood testing method that makes it possible to obtain information such as the amount of DNA with mutation derived from tumor cells and the number of replications per unit volume, and to accurately and easily perform quantitative analysis of the progression of tumors and the therapeutic reactivity without placing any heavy burden on a patient.

The above-described object can be achieved by the present invention as described below.

Through devoted studies the inventors of this invention discovered that it is possible to diagnose the progression of tumors accurately and quantitatively by using, as a marker, changes in DNA or RNA specific for tumor cells, and by calculating a relative ratio between a mutation factor amount derived from the tumor cells and the normal factor amount with no mutation among DNA and RNA existing in the blood plasma.

The blood testing method of this invention is characterized in that it comprises the steps of: detecting a mutation factor derived from tumor cells and a normal factor with no mutation in a sample containing human plasma with the mutation factor; and calculating a relative ratio between the mutation factor amount and the normal factor amount.

The blood testing method of this invention is also characterized in that it comprises the steps of: separating, using the PCR-SSCP method, a mutation factor derived from tumor cells from a normal factor with no mutation in a sample containing human plasma with the mutation factor, and then detecting the mutation factor and the normal factor; and calculating a relative ratio between the mutation factor amount and the normal factor amount.

Moreover, the blood testing method of this invention is characterized in that it comprises the steps of: amplifying a sample, which contains human plasma with a mutation factor derived from tumor cells, by means of the competitive PCR method using a labeled primer, then separating the mutation factor from a normal factor with no mutation using the SSCP method, and then detecting the labeled part; and calculating a relative ratio between the mutation factor amount and the normal factor amount.

Furthermore, the blood testing method of this invention comprises the steps of: electrochemically or optically detecting a reaction product obtained by hybridization between a sample, which contains human plasma with a mutation factor derived from tumor cells, and a probe which has a base pair part complementary to DNA fragments or RNA fragments that contain mutation derived from tumor cells; electrochemically or optically detecting a reaction product obtained by hybridization between the sample and a probe which has a base pair part complementary to DNA fragments or RNA fragments that do not contain the mutation; and calculating a relative ratio between the mutation factor amount and the normal factor amount.

The mutation factor amount may be the number of mutated DNA fragments or mutated RNA fragments specific for tumor cells. There is no particular limitation on these mutated DNA fragments or mutated RNA fragments as long as they are specific for tumor cells. Examples of such mutated DNA fragments or mutated RNA fragments include DNA fragments or RNA fragments with mutations such as K-ras gene mutations or p53 gene mutations.

The normal factor amount may be the sum of the number of DNA fragments derived from normal cells and the number of DNA fragments that are derived from tumor cells and contain no mutation, or the sum of the number of RNA fragments derived from normal cells and the number of RNA fragments that are derived from tumor cells and contain no mutation.

The blood testing method of this invention is characterized in that it comprises the step of detecting the number of DNA fragments or the number of RNA fragments that contain mutation derived from tumor cells, per unit volume of a sample containing human plasma with a mutation factor derived from tumor cells.

Moreover, this invention provides a testing chip, on which a probe having a base pair part complementary to DNA fragments or RNA fragments that contain mutation derived from tumor cells, and a probe having a base pair part complementary to DNA fragments or RNA fragments that do not contain the mutation, are immobilized.

This invention provides a testing device comprising the above-described testing chip.

### BRIEF DESCRIPTION OF THE DRAWINGS

FIG. 1 is a perspective view illustrating the entire structure of the testing device of the present invention.

FIG. 2 is a perspective view illustrating the entire structure of the testing chip of the present invention.

FIG. 3 is a graph showing the progression of CEA and plasma DNA of a colon cancer patient of Example 1.

### BEST MODE FOR CARRYING OUT THE INVENTION

The blood testing method, the testing chip, and the testing device according to the embodiments of the present invention are hereinafter described with reference to the attached drawings. However, the drawings are only to illustrate the examples of this invention and, therefore, are not intended to define the limits of this invention.

(Blood Testing Method)

The blood testing method of this invention is characterized in that it comprises the steps of: detecting a mutation factor derived from tumor cells and a normal factor with no mutation in a sample containing human plasma with the mutation factor; and calculating a relative ratio between the mutation factor amount and the normal factor amount.

Tumor cells are a part of human somatic cells that have grown excessively. Examples of tumor cells include: malignant tumors including cancer such as stomach cancer and liver cancer, lymphoma, sarcoma such as osteosarcorma, and leukemia; and benign tumors.

Mutation refers to a changed part of the DNA or RNA specific to the tumor cells. Specifically, the mutation refers to the somatic cells whose genome base sequence has mutated in a manner specific to the tumor cells (for example, when the base sequence has been lost, inserted, or replaced). Somatic cells having changes such as the methylation of DNA may also be considered mutation.

Human plasma refers to a part of human blood with blood cells removed therefrom. Concerning the testing method of this invention, any one of blood, blood plasma, blood serum, or any of these to which pretreatment is applied with specified conditions may be used as a detection object (sample). For example, the mutation factor and the normal factor in the blood plasma may be detected, or the mutation factor and the normal factor in the blood serum, which is obtained by further removing a blood coagulation factor from the blood plasma, maybe detected, or the mutation factor and the normal factor in the blood may be detected. Alternatively, after pretreatment is applied to the blood plasma or the blood serum with specified conditions, the mutation factor and the normal factor contained in the treated solution may be detected.

The relative ratio between the mutation factor amount and the normal factor amount can be found according to formula (1) below. In the following formula (1), the number of mutated DNA fragments refers to the number of DNA fragments derived from tumor cells having mutation, while the number of normal DNA fragments refers to the sum of the number of DNA fragments derived from normal cells and the number of DNA fragments derived from tumor cells that have no mutation.

Relative Ratio the Number of Mutated DNA fragments/the Number of Normal DNA Fragments Formula (1):

When the number of tumor cells increases or medical treatment causes cytolysis of the tumor cells, the above-mentioned ratio increases. On the other hand, when the tumor cells have regressed and the amount of DNA discharged into the blood plasma is lessened, the above-mentioned ratio decreases.

Since the numeric value of the relative ratio between the mutation factor amount and the normal factor amount is calculated as described above, it is possible to accurately test the progression of cancer even if there are variations in DNA concentration or RNA concentration in the blood plasma among individuals.

The mutation derived from the tumor cells includes all mutations existing in genomes of the tumor cells. Examples of the mutation include K-ras gene mutation or p53 gene mutation, which can be observed in a high frequency in cancer patients. It is possible to detect the progression of cancer quantitatively and accurately at frequent intervals by calculating the relative ratio between the amount of DNA fragments derived from the tumor cells with the above-mentioned mutation, and the amount of DNA fragments with no mutation. It is also possible to test the progression of cancer quantitatively and accurately at frequent intervals by calculating the relative ratio between the amount of RNA fragments derived from the tumor cells with the above-mentioned mutation, and the amount of RNA fragments with no mutation.

The above-described testing method may be conducted by first separating, using the PCR-SSCP method, the mutation factor derived from tumor cells from the normal factor with no mutation in the sample containing human plasma with the mutation factor, and then detecting the mutation factor and the normal factor, and then calculating the relative ratio between the mutation factor amount and the normal factor amount.

For example, the sample which contains human plasma with the mutation factor derived from tumor cells, may first be amplified by the competitive PCR method using a primer labeled with, for example, a fluorescent material. After the amplification, the mutation factor maybe separated from the normal factor with no mutation using the SSCP method, and the labeled part may then be detected, and the relative ratio between the mutation factor amount and the normal factor amount may be detected. After the amplification of the sample using the competitive PCR method using the same primer in the state where the mutation factor and the normal factor coexist, the mutation factor and normal factor amounts may be measured respectively and the relative ratio may then be calculated. The method of this invention is effective because the existence ratios of these factors before and after the amplification using the competitive PCR method are the same.

Moreover, instead of using the labeled primer, the mutation factor and the normal factor may be labeled before or after the separation of the mutation factor from the normal factor using the SSCP method, and these factors may be detected, and the relative ratio between the mutation factor and the normal factor may be calculated.

Furthermore, a testing method may be employed that comprises the steps of: electrochemically or optically detecting a reaction product obtained by hybridization between a sample, which contains human plasma with a mutation factor derived from tumor cells, and a probe which has a base pair part complementary to DNA fragments or RNA fragments that contain mutation derived from tumor cells; electrochemically or optically detecting a reaction product obtained by hybridization between the sample and a probe which has a base pair part complementary to DNA fragments or RNA fragments that do not contain the mutation; and calculating a relative ratio between the mutation factor amount and the normal factor amount.

Concerning the detecting step of using the probe which has a base pair part complementary to DNA fragments or RNA fragments that contain the mutation and the detecting step of using the probe which has a base pair part complementary to DNA fragments or RNA fragments that do not contain the mutation, either one of the detecting steps may be conducted first and the other afterward, or both detecting steps may be conducted at the same time. In other words, a detecting chip on which the probe for the former detecting step is immobilized and a detecting chip on which the probe for the latter detecting step is immobilized, may be used respectively to conduct the detections in sequence, or a testing chip on which both the probes are immobilized may be used to conduct the detections at the same time.

The probe is used to search for the DNA fragments or RNA fragments in the sample. Examples of the probe to be used include, other than DNA with a base pair part complementary to the DNA fragments or RNA fragments, PCR products, oligonucleotide, mRNA, cDNA, PNA (peptidic nucleic acid), or LNA (locked nucleic acid, which is a trademark owned by Proligo LLC).

In order to detect the reaction product photochemically, it is possible to conduct the PCR by using a primer (fluorescent primer) made of nucleotide modified with a fluorescent material, and to analyze the obtained product by using an automatic DNA sequencer.

It is also possible to optically detect the reaction product by labeling DNA or RNA, as the sample, with a fluorescent material, conducting hybridization and then reading the labeled DNA or RNA with a fluorescence scanner, thereby analyzing the signal strength. The fluorescent-labeled sample binds with a DNA probe with a complementary sequence, and the obtained sample is then measured quantitatively as a fluorescent image. The measurement is facilitated by using a DNA chip that is made by placing, on a chip, a multiplicity of probes in an array.

On the other hand, in order to conduct the quantitative detection with a high degree of precision, it is desirable to conduct the detection electrochemically. The electrochemical detection is performed by, for example, detecting changes in the current value flowing through the reaction product.

When the electrochemical detection is performed, it is desirable that the reaction product be caused to react with an intercalator, thereby measuring changes in the detected current value.

Examples of the intercalator include ferrocene, catecholeamine, a metal bipyridine complex, a metal phenanthrene complex, viologen, or a threading intercalator compound into which any of the above-listed compounds is introduced. A particularly preferred type of intercalator is a ferrocene threading intercalator compound.

The intercalator intrudes into double-stranded layers and forms a kind of charge-transfer complex. The intercalation causes changes in the current value running through electrodes. Such changes in the current value are caused by an oxidation-reduction reaction of the intercalator bound with the double strands. By detecting this current value, it is possible to measure the amount of the mutation factor derived from tumor cells (for example, molar concentration of the DNA fragments derived from tumor cells).

As described above, it is possible to test the progression of tumors quantitatively and accurately at frequent intervals by causing the sample and the probe to react against one another and by detecting the obtained reaction product.

Another testing method of this invention is to detect the number of DNA fragments or RNA fragments, which contain mutation derived from tumor cells, per unit volume of a sample containing human plasma with a mutation factor derived from tumor cells.

By detecting the number of mutated DNA fragments or RNA fragments contained per unit volume (e.g., 1 ml of blood plasma) of the sample, it is possible to test the progression of tumors quantitatively and accurately. Specifically, not only is the DNA amount in the blood plasma measured, but the number (or concentration) of the mutated DNA fragments or RNA fragments is also measured. Therefore, it is possible to accurately test the progression of tumors without being influenced by variations in the DNA concentration or RNA concentration in the blood plasma among individuals.

Concerning the above-described testing method, the testing object is not tissue, but blood, and the test can be conducted easily and quantitatively by taking blood from patients. Accordingly, it is possible to conduct the test frequently without placing any heavy burden on patients and to measure the progression of tumors with time.

(Probe)

An explanation is given below about the probe used in the above-described testing method.

The probe used in the above-described testing method has a base pair part complementary to the DNA fragments or RNA fragments contained in the mutation factor derived from tumor cells. The immobilized probe may have a base pair part complementary to the DNA fragments or RNA fragments with K-ras or p53 gene mutations.

There is no particular limitation on the type of probe so long as it has the complementary base pair part as described above. Examples of the probe to be used may be either of the following: DNA obtained by breaking, with a restriction enzyme, DNA which is extracted from a biotic sample, and by refining the obtained DNA by means of, for example, separation such as electrophoresis; or chemically synthesized DNA. The sequence of the probe should be decided in advance, depending on the DNA fragments (or object fragments) contained in the mutation factor.

(Testing Chip and Testing Device)

The testing chip and the testing device that are used in the above-described electrochemical testing method are hereinafter described.

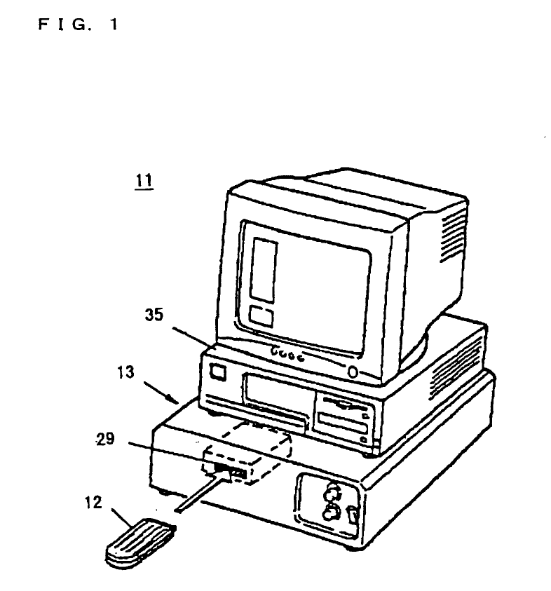

FIG. 1 is a perspective view illustrating the entire structure of the testing device of this invention. Referring to FIG. 1, a testing device 11 of this invention has a testing chip 12 and an insertion hole 29 into which the testing chip 12 can be inserted. This testing device 11 is composed of a measuring device 13, which can detect the probe electrochemically and can also electrochemically detect the double strands generated by hybridization, and a personal computer 35.

As for the electrochemical detecting means, a cyclic voltammetry, a differential pulse voltammetry, a potentiostat or the like can be used.

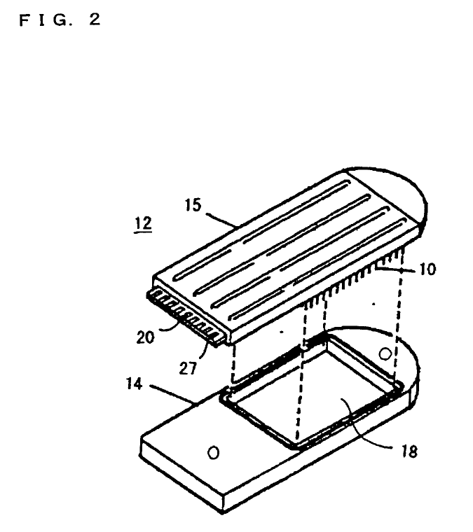

FIG. 2 illustrates the structure of the detecting chip 12. As shown in FIG. 2, the detecting chip 12 has electrodes 10, on which the probe is immobilized, and a common electrode (not shown in FIG. 2) which is the counter electrode of the electrodes 10. The detecting chip 12 can electrochemically detect the double strands formed by hybridization between the probe and the DNA fragments by applying a voltage between the electrodes 10 and the common electrode.

Specifically, the detecting chip 12 is composed of a frame 14, which has a depression 18 formed in its midsection, and a main body 15 which is mounted on the frame 14 in an attachable or detachable manner. The depression 18 is designed to be filled with a solution (including the probe, the sample, the intercalator, and a wash). In the area of the main body 15 that corresponds to the depression 18 of the frame 14, a multiplicity of pin electrodes 10 are mounted protuberantly and uniformly. The solution containing the probe is injected into the depression 18, and the probe is then immobilized through thio (SH) groups existing in its molecules to the surface of the pin electrodes 10.

Subsequently, operations for injection of the specified solution into, and washing of the depression 18 are conducted in sequence, and the hybridization and the intercalation are performed. Then, the detecting chip 12 is inserted into the insertion hole 29, a terminal 27 for the common electrode and a terminal 20 for the respective pin electrodes are connected to receiving terminals of the measuring device and then connected through a selecting switch to a voltage circuit. As a weak voltage is applied to the common electrode and the respective pin electrodes 10, a very weak current flows between the part labeled by the intercalator and the pin electrodes 10 through the medium of the voltage circuit and the common electrode. Detection is carried out through the detection of this electric current.

As for the electrodes for this invention, any electrodes can be used as long as the probe can be immobilized to such electrodes. Preferred examples of the electrodes include gold, glassy carbon, and carbon.

Known methods are used as the method for immobilizing the probe to the electrodes. For example, if the electrodes are gold, the thiol (SH) group is introduced to the probe body, and the probe is then combined with the electrodes through the medium of gold-sulfur coordinate bonds between gold and sulfur. The method for introducing the thiol group to the probe body is described in the following references: Mizuo Maeda, Koji Nakano, Shinji Uchida, and Makoto Takagi, “Chemistry Letters,” 1805-1808 (1994); and B. A. Connolly, “Nucleic Acids Rs.,” 13, 4484 (1985).

It is possible to conduct the test easily by using the testing chip and the testing device to test the blood taken from patients.

### EXAMPLES

Examples of this invention are hereinafter described. Needless to say, however, this invention is not limited to the following examples.

### Example 1

DNA was refined by separating blood plasma by means of centrifugal separation from blood taken from a colon cancer patient, and by treating the obtained blood plasma with proteinaseK, phenol, and chloroform. The refined DNA derives from in vivo normal cells and tumor cells.

By using this DNA as a template, the area containing DNA mutation (K-ras gene mutation) which can be recognized only in the tumor cells was amplified by the competitive PCR method. A primer labeled with a fluorescent material was used. The PCR method was conducted by causing denaturation at a temperature of 95° C. for 10 minutes, and then causing the reaction by repeating 45 to 50 cycles at 94° C. temperatures for 30 seconds, and at 60° C. for 30 seconds, and then by continuing the reaction at a temperature of 72° C. for 5 minutes, thereby causing amplification.

The obtained PCR product contained mutated DNA fragments with mutation and normal DNA fragments with no mutation. The mutated DNA fragments were generated by amplifying DNA derived from tumor cells. The normal DNA fragments were generated by amplifying DNA derived from normal cells and from normal alleles in tumor cells. Since these DNA fragments were amplified by the competitive PCR method, the existence ratio between the mutated DNA fragments and the normal DNA fragments in the DNA template used was reflected in the existence ratio between the mutated DNA fragments and the normal DNA fragments in the amplified PCR product.

The amplified DNA fragments were separated into the mutated DNA fragments and the normal DNA fragments using the SSCP method through utilization of an automatic sequencer. The existence ratio between the mutated DNA fragments and the normal DNA fragments was measured according to their signal strength.

The numerical values for equations “M/M+W” and “M/W” were respectively obtained when “M” refers to the signal strength of the mutated DNA fragments and “W” refers to the signal strength of the normal DNA fragments.

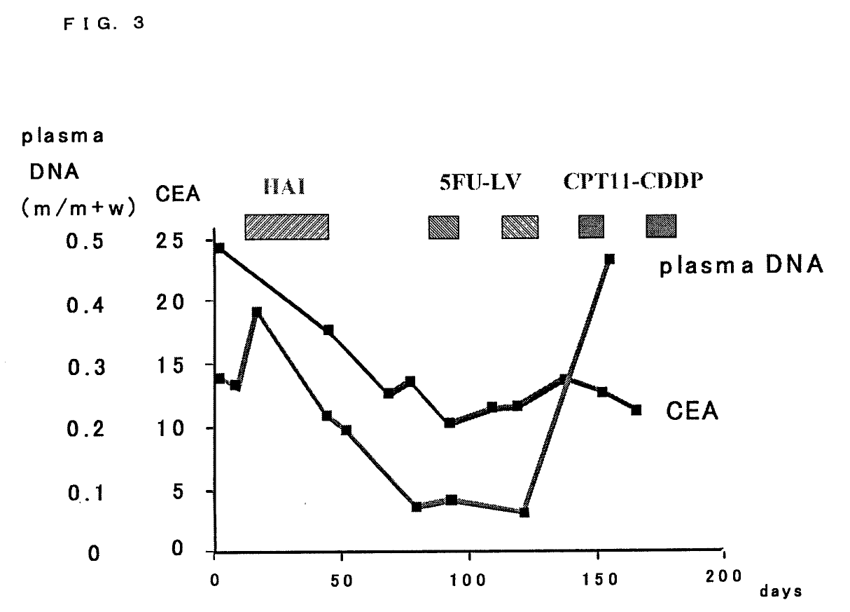

FIG. 3 shows the CEA progression (carcinoembryonic antigen) and the progression of the M/M+W signal ratio of plasma DNA when the colon cancer patient underwent chemotherapy using carcinostatic drugs. As shown in FIG. 3, when the progressions of the numerical values were examined with time, the M/M+W signal ratio decreased to approximately 0.1 or less until about 120 days had elapsed from the date of the chemotherapy. This progression of the M/M+w signal ratio was parallel to that of CEA.

After 120 days had elapsed, chemotherapy was conducted on cancer cells that had grown again. This chemotherapy was specifically effective on the cancer cells, and the dead cancer cell DNA flowed into the blood, thereby causing the M/M+W signal ratio to increase sharply.

Accordingly, it was confirmed that the M/M+W signal ratio reflected the progression of cancer and the therapeutic effect.

It was also confirmed that the M/M+W signal ratio drastically decreased when the tumor of another colon cancer patient was surgically removed.

### Example 2

Example 2 was different from Example 1 in that an allele-specific primer was used for the refined plasma DNA, and the real-time PCR method was employed to measure a relative ratio between the number of the mutated DNA fragments and the number of the normal DNA fragments. Also in this case, it was possible to estimate the progression of tumors by numerically expressing the ratio (or concentration ratio) between the number M of the mutated DNA fragments and the number W of the normal DNA fragments.

### Example 3

In Example 3, the refined DNA was made to react to the electrodes on which the probe having a base pair part complementary to the mutated DNA fragments was immobilized. The intercalator was injected into a container, hybridization was performed to form double strands of the DNA fragments, and the double-stranded DNA fragments were then made to react specifically. Changes in the detected current value were measured and the molar concentration of the mutated DNA fragments was found.

Also in this example, the ratio (or concentration ratio) between the number M of the mutated DNA fragments and the number W of the normal DNA fragments was found by measuring the changes in the detected current value, thereby making it possible to surmise the progression of tumors.

### Example 4

The blood plasma was separated in the same manner as in Example 1, thereby collecting RNA and conducting the analysis. Specifically, RNA was collected from 1 ml of the blood plasma using the AGPC (acid guanidinium thiocyanate-phenol-chloroform) method. This RNA was used with reverse transcriptase to synthesize cDNA. This cDNA was used as a template to conduct the PCR with a primer for CDNA of the K-ras gene. The amplified CDNA was separated into mutated cDNA fragments and normal cDNA fragments using the SSCP method that uses the automatic sequencer, and the existence ratio between the mutated cDNA fragments and the normal cDNA fragments, was measured according to their strength.

As a result, it was confirmed that RNA also existed in the blood plasma. This means that the plasma RNA, like the plasma DNA, can be used as a tumor marker or for judgment of the therapeutic effect. As described above, it was possible to effectively conduct the testing by measuring the number of replications per unit volume of the blood plasma by employing the quantitative PCR.

This invention can be carried out in other various manners without deviating from the spirit and primary characteristics of the invention. Accordingly, the above-described examples are for the purpose of illustration only and should not be interpreted in any limited manner. The scope of this invention is indicated in the scope of the claims and shall not be bound by the main text of this specification.

### INDUSTRIAL APPLICABILITY

The blood testing method, the testing chip, and the testing device of this invention are designed to detect the relative ratio between the mutation factor derived from tumor cells and the normal factor with no mutation in the sample, which contains human plasma with the mutation factor derived from tumor cells, or to detect the number of DNA fragments containing mutation, thereby making it possible to quantitatively and accurately test the progression, metastasis, therapeutic effect, or recurrence of tumors at frequent intervals.

This invention makes it possible to quantitatively, accurately and easily realize, for example, early discovery of malignant tumors, post-operation tracing of tumors, research into the effects of drug administration or chemotherapy, and the selection of appropriate therapies.

What is claimed is:

1. A blood testing method comprising the steps of:
detecting a mutation factor derived from tumor cells and a normal factor with no mutation in a sample containing human plasma with the mutation factor; and
calculating a relative ratio between the mutation factor amount and the normal factor amount.

2. A blood testing method comprising the steps of:
separating, by the PCR-SSCP method, a mutation factor derived from tumor cells from a normal factor with no mutation in a sample containing human plasma with the mutation factor, and then detecting the mutation factor and the normal factor; and
calculating a relative ratio between the mutation factor amount and the normal factor amount.

3. A blood testing method comprising the steps of:
amplifying a sample, which contains human plasma with a mutation factor derived from tumor cells, by means of the competitive PCR method that uses a labeled primer, then separating the mutation factor from a normal factor with no mutation using the SSCP method, and then detecting the labeled part; and
calculating a relative ratio between the mutation factor amount and the normal factor amount.

4. A blood testing method comprising the steps of:
electrochemically or optically detecting a reaction product obtained by hybridization between a sample, which contains human plasma with a mutation factor derived from tumor cells, and a probe which has a base pair part complementary to DNA fragments or RNA fragments that contain mutation derived from tumor cells;
electrochemically or optically detecting a reaction product obtained by hybridization between the sample and a probe which has a base pair part complementary to DNA fragments or RNA fragments that do not contain the mutation; and
calculating a relative ratio between the mutation factor amount and the normal factor amount.

5. The blood testing method according to claim 1, wherein the mutation factor amount is the number of DNA fragments or RNA fragments with K-ras gene mutation or p53 gene mutation.

6. The blood testing method according to claim 1, wherein the normal factor amount is the sum of the number of DNA fragments derived from normal cells and the number of DNA fragments that are derived from tumor cells and contain no mutation, or the sum of the number of RNA fragments derived from normal cells and the number of RNA fragments that are derived from tumor cells and contain no mutation.

7. A blood testing method comprising the step of detecting the number of DNA fragments or the number of RNA fragments that contain mutation derived from tumor cells, per unit volume of a sample containing human plasma with a mutation factor derived from tumor cells.

8. A testing chip used for the blood testing method of claim 4, wherein a probe having a base pair part complementary to DNA fragments or RNA fragments that contain mutation derived from tumor cells, and a probe having a base pair part complementary to DNA fragments or RNA fragments that do not contain the mutation, are immobilized.

9. A testing device comprising the testing chip of claim 8.
